(19) United States
(12) Patent Application Publication
(10) Pub. No.: US 2025/0160800 A1
(43) Pub. Date: May 22, 2025

(54) IMAGING DIMENSIONAL CRYSTAL STRUCTURES INDICATIVE OF PHYSIOLOGICAL STATES

(71) Applicant: Ovulio Corp., Dover, DE (US)

(72) Inventors: Serhii Zatsarynin, Cambridge, MN (US); Ihor Kovalenko, Kyiv (UA)

(21) Appl. No.: 18/747,198

(22) Filed: Jun. 18, 2024

Related U.S. Application Data
(60) Provisional application No. 63/601,048, filed on Nov. 20, 2023.

(51) Int. Cl. A61B 10/00; G01N 33/487; G16H 40/67; G16H 50/20; G16H 50/70

(52) U.S. Cl. CPC A61B 10/0012; G01N 33/487; G16H 40/67; G16H 50/20; G16H 50/70; A61B 2010/0025; G01N 2333/723

(57) ABSTRACT

The invention relates to a method and an electronic autonomous device for prediction and testing physiological conditions associated with hormone levels in women. This innovation involves obtaining high-contrast images of dimensional crystal structures, including those in dried samples of a woman's mucous fluid, using a built-in magnification optical system with frontal illumination. Prediction and testing of physiological conditions of women is based on detecting the presence of fern-like structures in dried sample of mucus fluids by obtaining a dimensional image of the crystal structure of the sample and comparing the density of the crystals with the reference samples using the device with spaced artificial light sources and an opaque object glass, which allows obtaining detailed images of crystal structures. According to the invented method, a device can also be used to image flat objects like test strips or barcodes.

### CROSS-REFERENCE TO RELATED APPLICATIONS

The present application claims the benefit of U.S. Provisional Application No. 63/601,048, filed Nov. 20, 2023, which is incorporated herein in its entirety by reference.

### TECHNICAL FIELD

The present disclosure is directed to method and systems for imaging dimensional crystal structures in bodily fluids that are indicative of physiological states, such as structures present in saliva samples to determining physiological states related to ovulation in women.

### BACKGROUND

In women, ovulation usually occurs in the middle of the menstrual cycle. The most fertile period lasts approximately four days before and one day after ovulation, often referred to as the “fertile window.” At this time, when the life expectancy of the sperm (about 5 days) and the egg (about 1 day) coincide, the probability of conception reaches its peak. Outside of this window, the chances of conception decrease rapidly, making a woman less likely to get pregnant during that menstrual cycle.

### SUMMARY

The present disclosure relates to methods and systems, such as an electronic autonomous device, for determining physiological states related to ovulation based on the level of estrogen in a woman's mucosal fluid. While useful in tracking ovulation cycles, i.e., predicting the time of ovulation, the disclosed systems and technique also enable early detection of pregnancy during the luteal phase of the reproductive cycle, confirming healthy fetal development, indicating date of delivery, as well as tracking other fertility and gynecological issues that either cause or are otherwise indicated by a change in estrogen levels. Additionally, the systems and techniques describe herein may be used to determine physiological states related to detectable levels of other hormones, metabolites, or other biological fluids.

Various embodiments of the disclosure include a system configured to determine a physiological state of a woman associated with a hormone level that leads to an appearance of crystals having a characteristic fern shape in a dried sample of a mucous fluid of the woman. The system includes an optical sensor; a magnifying optical system; at least one of a frontal artificial light source and backlit artificial light source; a diffusion system; a memory including program code; processing circuitry configured to execute the program code of the memory; and an autonomous power management module. The processing circuitry is configured to detect, using the optical sensor, the presence of crystals in a sample by capturing an image of the crystal structure of the sample. The processing circuitry is further configured to predict the physiological state of a woman by comparing the crystal density with a reference database. The autonomous power management module is operatively coupled to the processing circuitry.

Various embodiments of the disclosure include a hand-held analyzer case for determining a physiological state of a woman. The hand-held case may include a first housing portion and a second housing portion. The first housing portion may include a support for receiving a saliva sample. The second housing portion may include an optical sensor, a magnifying optical system, a light source, a diffusion system, a processing unit, and a power source. The light source, diffusion system, magnifying optical system, and optical sensor may be disposed in an ordered columnar arrangement along a central vertical axis of the second housing portion. The ordered columnar arrangement is generally centered and vertically offset from the saliva sample.

Various embodiments of the disclosure are directed to a method of determining a physiological state of a woman associated with a hormone level that leads to an appearance of crystals having a characteristic fern shape in a dried sample of a mucous fluid of the woman. The method includes placing a sample of the mucous fluid in an optical path of an optical system of an autonomous device. The optical system includes an optical sensor, a magnifying optical system, at least one of a frontal artificial light source and a backlit artificial light source, and a diffusion system. The method further includes at least partially drying the sample; obtaining, by the optical system, a contrast image of the at least partially dried sample; identifying, by processing circuitry operatively coupled to the optical system, based on the contrast image, a presence of crystals in the sample; comparing, by the processing circuitry, the identified crystals to at least one reference image; predicting, by the processing circuitry, based on the comparison, the physiological state of the woman; and generating, by the processing circuitry, an alert indicative of the physiological state, wherein the alert is receivable by a human or a machine.

A feature and advantage of embodiments is a portable and autonomous device that can provide information about a physiological state, such as an indication of ovulation of a woman. A feature and advantage of embodiments is using samples, such as mucous fluids, that are readily obtainable without using invasive techniques or procedures for the determination of the physiological state. A feature and advantage of embodiments is early detection of physiological state that may require treatment.

### BRIEF DESCRIPTION OF THE DRAWINGS

The disclosure can be understood in consideration of the following detailed description of various embodiments in connection with the accompanying drawings.

FIG. 1 is a conceptual diagram illustrating levels of estrogen, progesterone, and luteinizing hormone during the female human menstruation cycle.

FIGS. 2A-2C are conceptual diagrams illustrating three typical types of saliva crystallization at different periods of a woman's menstrual cycle.

FIG. 3A is a conceptual diagram illustrating a perspective view of saliva crystallization analyzer device according to embodiments.

FIG. 3B is a side view of the saliva crystallization analyzer device of FIG. 3A.

FIG. 3C is a conceptual diagram illustrating embodiments of a saliva crystallization analyzer device.

FIG. 3D is a conceptual diagram illustrating embodiments of a saliva crystallization analyzer device.

FIGS. 4A through 4C are conceptual diagrams illustrating the sample of dried saliva with fern-like crystals for different imaging conditions.

FIGS. 5A and 5B are conceptual diagrams illustrating a backlit and front illumination of a test strip.

FIG. 6 is a conceptual diagram illustrating an example computing system of a saliva crystallization analyzer device.

FIG. 7 is a method of operation of saliva crystallization analyzer device according to embodiments.

FIG. 8 is a conceptual diagram illustrating an example connected saliva crystallization analyzer device according to embodiments.

### DETAILED DESCRIPTION

The present disclosure makes use of a phenomenon that occurs in a dried sample of a woman's mucous fluid, such as saliva or cervical mucus. It manifests itself as a distinct fern-like pattern resulting from the crystallization of sodium chloride and potassium chloride on mucus fibers to form a complex, heterogeneous network. This crystallization, known as “ferning,” is a consequence of elevated levels of estrogen in the body. As early as 1945, Georgios Papanicolaou observed the formation of crystals when a drop of cervical mucus dried on a slide. Further studies by Rydbergm and Madsen (Rydbergm, E. and Madsen, V. 1948. Acta Obst. And Gynec.

Scandinay) confirmed that these crystals, often called salt crystals, were the result of the presence of mucin, a glycoprotein containing acidic polysaccharides present in the mucosal secretion. In 1954, Zondek and Rosin further expanded (Zondek, B. and Rozin, S. 1954 Obst. and Gynec) on this phenomenon by establishing that crystallization occurs not only in cervical mucus but also in various mucosal secretions and body fluids.

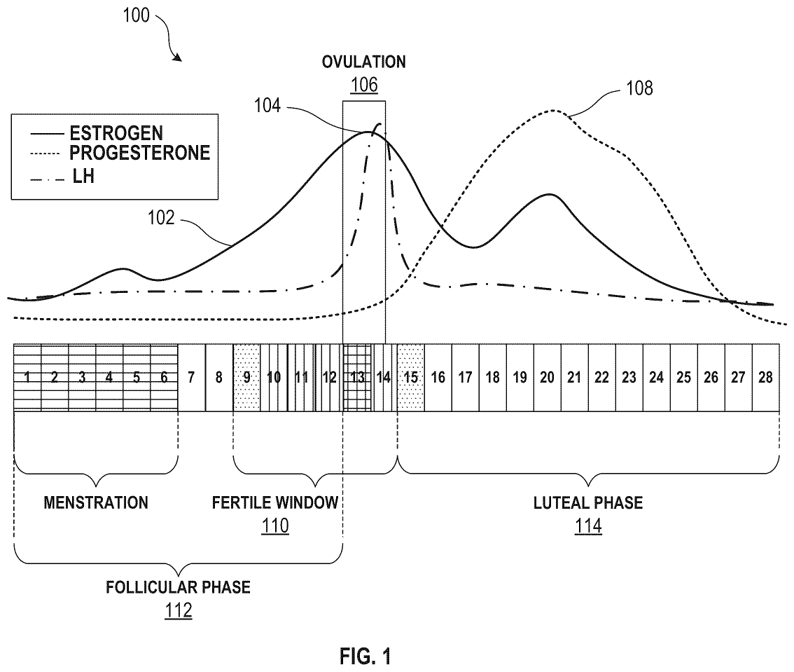

Understanding the complex processes that underlie the female reproductive system is important for couples seeking to maximize their chances of conceiving. Scientific research has provided valuable insights into the factors that influence fertility and the importance of monitoring physiological changes to determine the optimal time to conceive. FIG. 1 depicts levels of three female hormones during a typical 28 day menstruation cycle 100. Estrogen, a key hormone in the female reproductive cycle, plays a key role in signaling the onset of ovulation and peak fertility. Studies have consistently shown that estrogen levels 102 peak 104 just before ovulation 106, creating an environment conducive to successful fertilization. As shown in FIG. 1, this generally occurs between the 12th and 15th days of the cycle 100. The increase in estrogen levels 102 causes various physiological changes in the female body, in particular, the cervical mucus becomes more favorable for sperm, facilitating their way to the egg.

Around the time of ovulation 106, another hormone, progesterone, also increases, as illustrated by the dash line 108 in FIG. 1. Increased levels of progesterone 108 act as a natural contraceptive mechanism in the event that conception does not occur. This well-researched phenomenon protects against multiple fertilizations that can potentially lead to complications.

Accurate information about the maturation of the egg is crucial to determine the exact window for intercourse. This is not only a matter of timing, but also a matter of optimizing the conditions for conception. Studies emphasize that the probability of successful fertilization is highest during the fertile window, which includes approximately four days before and one day after ovulation. During this period, the life expectancy of the sperm (approximately 5 days) and the egg (approximately 1 day) coincide, offering a narrow but favorable time frame for fertilization.

Despite the complexity of the female reproductive system, fertility issues are a common concern for many couples. Studies have shown that approximately one in six women in the world has difficulty conceiving. This highlights the importance of developing reliable methods for tracking and predicting ovulation, as well as understanding the individual variations in the menstrual cycle that can occur from month to month.

The concept of the “fertile window” 110 is key to understanding fertility and choosing the time of fertilization. According to studies, the fertile window 110 is the period during which the chances of conception increase significantly. Accurately predicting this window 110 is of paramount importance for couples seeking to conceive and depends on an accurate estimate of estrogen levels and ovulation time. Ovulation 106, which occurs in the gap between the follicular phase 114 and the luteal phase 112, indicates the end of the fertile window 110 and generally occurs around day 13 or 14 of the cycle 100. As shown in FIG. 1, the fertile window 110 may be associated with a rise and peaking of estrogen levels. Further, a drop in estrogen levels 102, particularly following this 4-8 day rise in levels, may be associated with a transition from the fertile window 110 to the luteal phase 112.

The saliva fertility test, based on the observation of a characteristic fern-like patterns, is a non-invasive and effective method of predicting ovulation. Experimentally, it has been determined that the fern-like patterns can be observed at a magnification of more than 50 times. The characteristics of this pattern can change, providing valuable information about a woman's hormonal status. The characteristic of the fern-like pattern may include, but are not limited to crystal shape, crystal connections, crystal grouping or branching, angles of one or more ferns, fern density (e.g., a percentage of a predetermined area of an image with formed crystals). Such characteristics may be used separately or in any suitable combination for evaluation of an image.

Commercially available ovulation test strips check for levels of the luteinizing hormone (LH) 116 to determine when a woman is ovulating. Compare the described systems and techniques using the crystallization level of dried saliva to assess a woman's physiological state with commercial test strips for tracking ovulation, as shown in FIG. 1, the described systems and techniques are more informative by tracking the fertile window 110 lasting an average of 5 days, whereas commercial ovulation test strips give a positive result only 1 day on average.

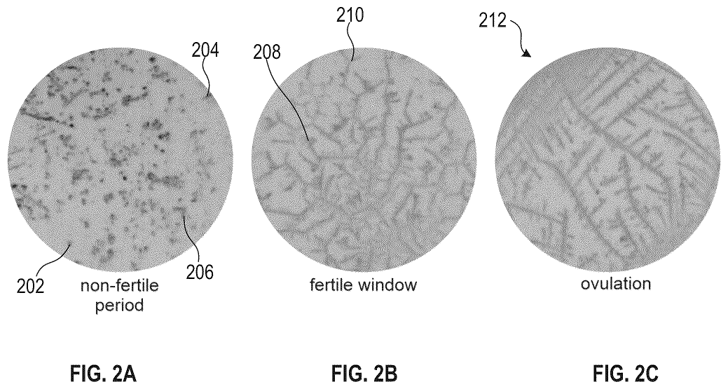

FIGS. 2A-2C show three typical types of saliva crystallization at different periods of a woman's menstrual cycle. The absence of a fern-like pattern in FIG. 2A, indicates limited visibility on the slide, often manifested as a few scattered dots 202, lines 204, or curved angular shapes 206 that may resemble spots or air bubbles. Partial fern formation, FIG. 2B is a transitional phase that occurs just before the onset of the fertile window 110. During this phase, the presence of crisscrossing lines 208 and angular shapes 210 gives a visual clue that the fertile period is approaching. Complete fern formation, shown in FIG. 2C, means the complete presence of fern-like patterns 212 throughout the slide, with the number of ferns 212 directly reflecting the estrogen levels in the sample. This method of estimating estrogen levels is well documented in the scientific literature, confirming its effectiveness in predicting ovulation and optimizing the timing of intercourse for couples seeking to become pregnant. These images may further be used to provide quantitative results of the estrogen hormone level in the mucous fluid.

In 2014, the U.S. Food and Drug Administration (FDA) granted approval for the saliva fern test to be used as a home test. Other home tests, such as the ovulation calendars and basal body temperature measurements, are only starting to be cleared by the FDA since 2021 due to their methodical challenges to provide clinically accurate predictions of ovulation time.

Most available saliva fertility testing devices allow users to visually observe the appearance of characteristic crystal shapes before ovulation. Typically, these devices are optical mini-microscopes and cannot digitize or assist in analyzing the presence and density of fern-like crystals. Some of these devices use mobile devices as a platform for image acquisition, processing, and visualization, but are not able to interpret the results or present an indication of physiological state to the user, which often makes it difficult to use the devices.

Some devices use partial or assisted image recognition algorithms to estimate the density of salivary fern crystals, but require user intervention when the saliva is completely dry, a process that can take 4 to 90 minutes, depending on environmental conditions. The quality of interpretation in such solutions depends on the user's skills and numerous factors, including camera positioning, focus, lighting, and drying time. Deviations in these factors can lead to inaccurate results.

An advantage of the systems and technique describe herein is to overcome the limitations of existing ovulation prediction methods. The systems and techniques enables an FDA-approved, non-invasive saliva fern test for home use that offers accurate predictions of ovulation time to help women get pregnant or, alternatively, can be used as a means of identifying a non-fertile period with a low chance of getting pregnant. In addition, the described systems and techniques do not require any knowledge or skills from the user to obtain a high-quality image, set up proper lighting, focus, blurring, or recognize the obtained results.

Another advantage of the systems and technique describe herein is addressing the shortcomings of existing methods for predicting the timing of labor. It can assist in forecasting the expected delivery time, as fern-like discharge typically becomes absent during the latter part of pregnancy but reappears approximately four weeks before delivery. More significantly, it can predict premature birth if the expected delivery time is early, allowing for timely medical intervention.

An additional purpose of the present disclosure is to overcome the problems associated with testing in early pregnancy, as the continued presence of fern crystals in saliva after the end of the fertile window may indicate pregnancy even before the absence of menstruation. Moreover, ferning may persist until near the end of the first trimester or until the plecenta fully develops, the lack of ferning may be indicative of problems associated with the development of the embryo or placenta.

Finally, this disclosure strives to resolve issues related to fertility and gynecological problems, as unexpected ferning or lack of ferning during expected periods of time may also signal health concerns or other health events, including, but not limited to, perimenopause, menopause, endometriosis, fibroids, polycystic ovarian syndrome, polyps, ovarian cancer, uterine cancer, breast cancer, heart disease, insulin resistance, dementia, genetic conditions (Turner syndrome and Fragile X), autoimmune diseases, pituitary gland disfunction, hypothalamic amenorrhea, combinations thereof, or the like.

In summary, scientific studies clearly emphasize the critical role of estrogen and progesterone in the female reproductive cycle and the importance of accurately predicting ovulation to increase the chances of conception. In addition, the saliva fertility test, with its distinctive fern-like pattern, offers a reliable and non-invasive way to monitor hormonal changes and accurately determine the fertile window. Backed by scientific evidence, this method provides couples with a valuable tool to increase fertility awareness and can greatly assist in choosing the best time to conceive.

The disclosure relates to the field of prediction and testing of physiological conditions of a woman associated with increased levels of hormones that lead to the appearance of crystals of the characteristic shape of fern leaves in a dried sample of a woman's mucous fluid, which includes a method for obtaining a contrast image of a sample of dried mucous fluid using an autonomous device, detecting the presence of crystals in the sample by taking a dimensional image of the crystal structure of the sample, predicting the physiological state of a woman by comparing the crystal density with the reference samples.

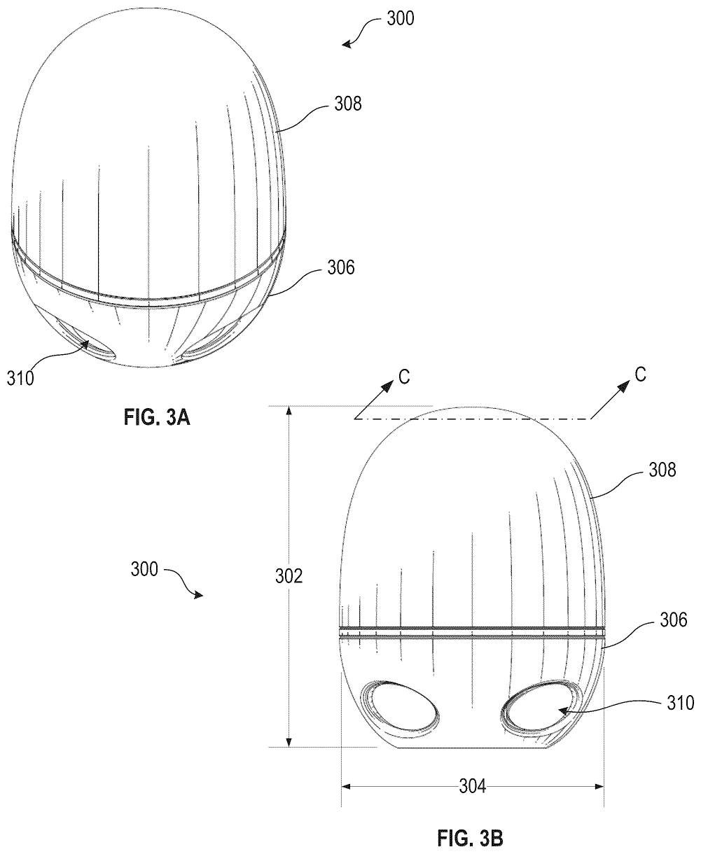

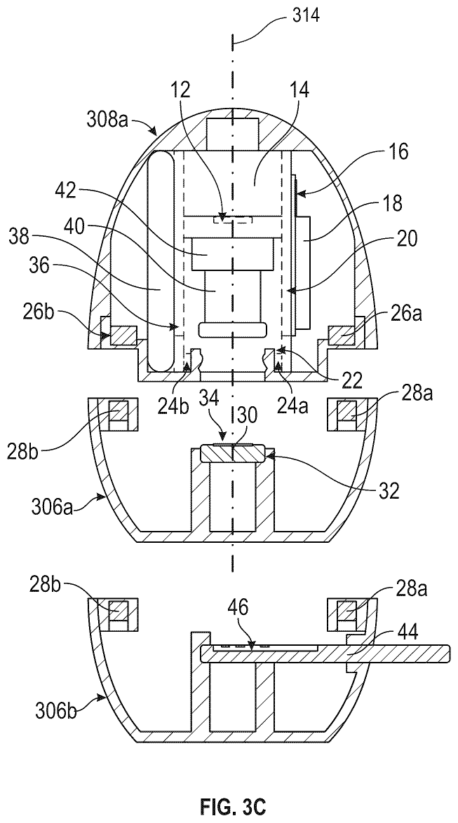

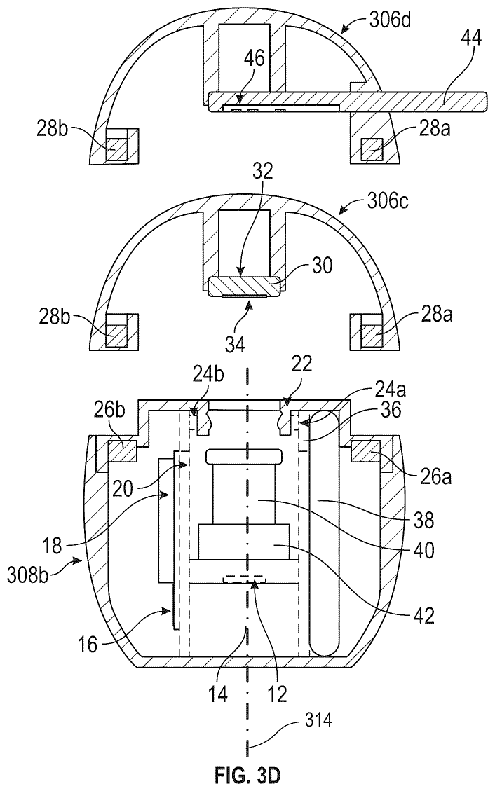

The disclosure also relates to a stand-alone analyzer device 300 as shown in FIGS. 3A-3D. Referring specifically to FIGS. 3A and 3B, in embodiments, the device 300 is a hand held device and may have a height 302 between 2 to 5 inches, and a width 304 between 1 to 4 inches. Device 300 is generally egg-shaped such that the height 302 is larger than the width 304. In other examples, device 300 may include other shapes. Device 300 is separable into a first housing portion, or cartridge, 306 and a second housing portion, or cartridge, 308. As discussed in detail below, first housing portion 306 may be used for receiving and supporting a sample, and second housing portion may contain electronic circuitry, power source(s), optical elements, and the like, for the operation of the device. In some embodiments, the first housing portion 306 may define one or more openings 310. Openings 310 may be of various shapes or sizes associated with their use. For example, larger openings may provide air flow for drying samples, whereas smaller openings or slots may be used for receiving test strips or the like. In embodiments, the second housing portion 306 may be longer than first housing portion 304.

FIG. 3C illustrates a sectional view of the device 300 with two alternate embodiments 306a, 306b of first housing portion 306. The second housing 304 of the device 300 includes at least one processor 18, an optical sensor 12 with a magnifying optical system 40 and at least one frontal artificial light source 24a (b), with diffusion system 22, at least one memory including program code, and an autonomous power management module 36 consisting of at least one rechargeable battery 38. First housing 306a of the device 300 is equipped with an opaque objective glass 30 for applying a sample of mucosal fluid 34. Alternatively, as illustrated by the first housing 306b, the device 300 is configured to receive optional indicator strip 44. The memory and program code are configured so that the device performs at least the following: imaging the dried mucosal fluid sample 34 or indication area 46 of test strip 44 using an optical sensor 12 through a magnifying optical system 40 using built-in artificial light source 24a (b) with diffusion system 22, detecting the presence of crystals in the sample by taking a contrast image of the crystal structure of the sample, determining the density of crystals in the sample, predicting a physiological condition of a woman associated with elevated hormone levels by comparing the density of crystals with the reference images for the first embodiment of first housing 306a or determining test result by taking a picture of indication area 46 of test strip 44 for the second embodiment of the first housing 306b.

The device operation is based on the described innovative method of obtaining high-quality contrast images, which differs from the existing methods in the principle of image formation. The optical devices for monitoring ovulation from dried saliva samples discussed above are low-magnification optical microscopes that work on the sample lumen on a glass slide using an artificial or natural light source, which can also be additionally focused by an auxiliary optical system. The described method is based on the principle of obtaining a contrast image of dimensional sample on an opaque 32 objective glass 30 using frontal illumination from several spaced artificial light sources 24a, 24b focused by an auxiliary optical system 22. This approach allows for a more contrasty image of dimensional structures because the frontal illumination, unlike the back illumination, creates a shadow on the surface of the objective glass. The use of several spaced artificial light sources makes it possible to neutralize the influence of the shape and direction of fern-liked crystals.

The first housing 306a,b has a support structure 308 generally centered on a central axis 310 of the device 312. The support structure 308 may be used to support the objective glass 30 according a first embodiment or a test strip 44 according a second embodiment.

Within the scope of the present disclosure, the device for capturing images of dried saliva for predicting the physiological state of a woman by comparing the crystal density with the reference samples is referred to as Option 1.

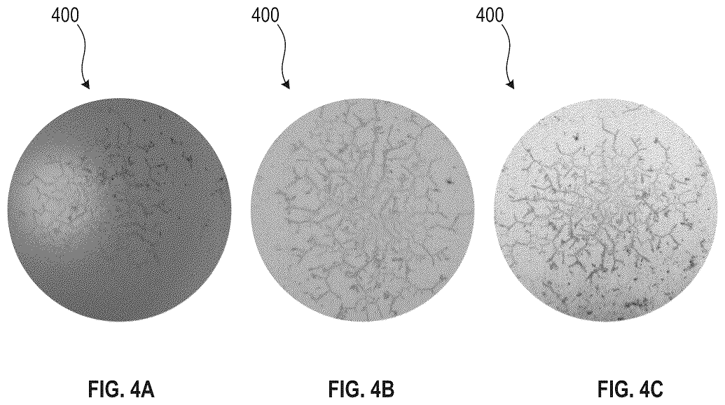

FIGS. 4A-4C show images of the same sample of dried saliva with fern-like crystals 400 for different imaging conditions, all other parameters and device settings being identical: FIG. 4A illustrates back illumination, lumen imaging—the principle of a conventional optical microscope; FIG. 4B illustrates front illumination, single light source; and FIG. 4C illustrates frontal illumination, two spaced artificial light sources. As illustrated, the image illustrated in FIG. 4C provides improved contrast, which may aid in automated or semiautonomous characterization of the fern-like crystals 400, compared to the imaging conditions in FIGS. 45A and 4B.

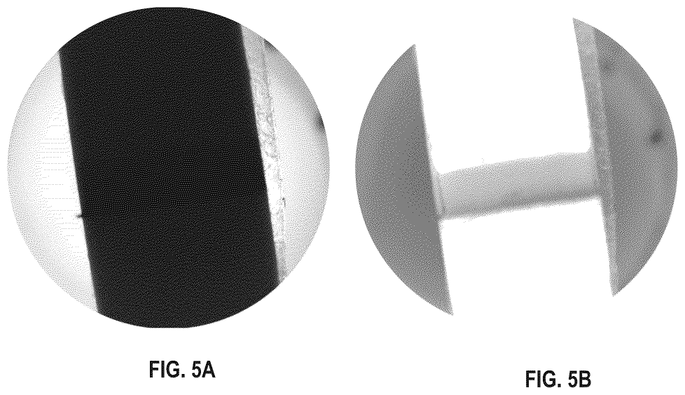

At the same time, this approach makes it possible to obtain conventional images of flat objects, such as test strips (FIG. 5B), barcodes, QR codes, which are in the focal area of the optical system, since the front illumination does not require the object under study to be transparent, unlike the back illumination (FIG. 5A). Accordingly, a device that uses this principle of image acquisition can also be used to obtain an image of the indication zone of test strips with its subsequent recognition and interpretation.

Within the scope of the present disclosure, the device for imaging flat objects, such as the indicator area of the test strips, barcodes, QR codes is referred to as Option 2.

The generalized approach allows us to design a universal instrument that can work with any of the above types of analyzes using replaceable cartridges that can be supplied. The test strip recognition cartridge Option 2 does not contain the test strip itself; it is proposed to use separate test strips that are inserted into the hole in the cartridge in such a way that the indicator zone of the strip is in the focal area of the optical system. The proposed approach makes it possible to reuse the cartridges without creating additional waste, which is also an advantage of the proposed device.

The proposed method of obtaining a sample image does not require a specific orientation in space or a specific orientation to external light sources. This makes it possible to manufacture the device shown in FIG. 3D, in which an object glass with a saliva sample or test strip placed above the optical system. This arrangement makes it possible to make the device more compact and/or to place a wireless charger in its lower part.

Within the scope of this disclosure, a device having the internal arrangement of elements shown in FIG. 3C is called Type 1. The device with the internal arrangement of elements shown in FIG. 3D is called Type 2. Accordingly, two variants of cartridge or first housing configurations 306a,b are also available for Type 2—for capturing images of dried saliva for predicting the physiological state of a woman by comparing the crystal density with the reference samples is referred to as Type 2 Option 1; for visualization of flat objects, such as the indicator area of test strips, barcodes, QR codes, called Type 2 Option 2.

Referring to FIGS. 3C and 3D, the second housing portion 308 of the analyzer case 300 is used to hold the internal elements and is part of the cartridge position fixation system so that the sample is within the focal length of the optical system and the front illumination. Cartridges or first housing portions 306a, b, c, d serve as an element of the device case that positions sample for analysis (or the indicator zone of the test strip) in a specific position relative to the magnifying optical system and to the front illumination system. Cartridge 306a is used for Type 1 Option 1, cartridge 306b is used for Type 1 Option 2, cartridge 306c is used for Type 2 Option 1, and cartridge 306d is used for Type 2 Option 2. The optical sensor 12 performs the function of converting light into electrical impulses. An optical sensor case 14 a structural part of the optical sensor 12 that provides its attachment to the other elements of the device and connection to the magnifying optical system. A wireless communication antenna 16 is used to emit electromagnetic waves into space. A CPU module 18 serves as the main computing part of the device, which performs the functions of managing, processing, storing, sending, and receiving of the data.

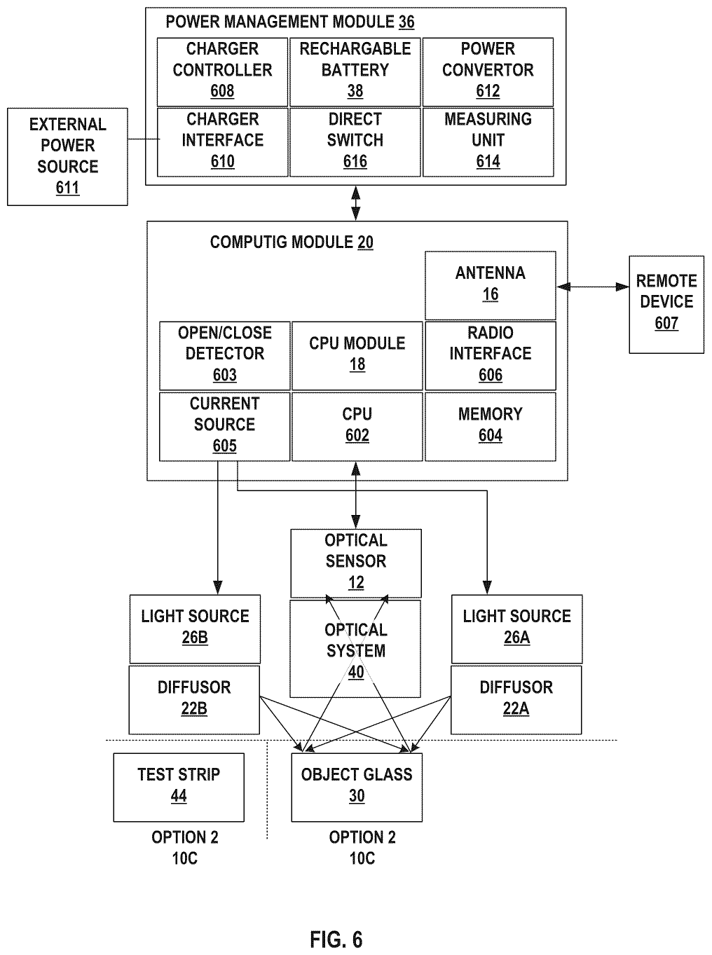

As illustrated in FIG. 6, computing module 20 includes, CPU module 18, CPU 602, open/close detector 603, static and dynamic memory 604, current source generator 605. and at least one radio interface 606. CPU 602 includes processing circuitry configured to execute one or more program instructions, such as instructions stored on memory 604. CPU module 18 may facilitate communication between PCU 602 and other components of computing module 20. Open/close detector 603 includes one or more electrical contacts configured to determine whether first housing portion and second housing portion are coupled or decoupled. Memory 604 includes computer-readable media in the form of volatile and/or nonvolatile memory and may be removable and/or non-removable is configured to store various types of data and executable computer-program instructions, such as, for example, include random access memory (RAM), read only memory (ROM), electronically erasable programmable read only memory (EE-PROM), flash memory, optical or magnetic storage devices, and/or other medium that can be used to store information and can be accessed by electronic devices. The radio interface 606 is configured to receive from CPU 602, e.g., via CPU module 18, data for transmission via antenna 16 using one or more standard communications protocols, such as, for example, Bluetooth, WiFi, NFC, cellular, or other protocols as known in the art. In some examples, antenna 16 may transmit to or receive from data remoted device 607, such as, for example, a smart phone, a tablet, a personal computer, a server, or a cloud based computing system. Elements of the CPU 18 may be positioned on a printed circuit board 20 within the second housing 308.

The power management module 36 may include a printed circuit board with at least one charge controller 608, at least one charging interface 610 configured to couple to an external power source 611, at least one voltage converter 612, measurement circuits 614, and direct switch 616. The power management module 36 may also include a battery 38. In embodiments, the battery 38 may be a rechargeable battery.

An optical front illumination system 22 is used to form the necessary light flux for optimal sample illumination. The optical front illumination system 22 may include artificial light sources 24a,b which are used to generate light flux. Positioned in line with the illumination system 22 is a magnifying optical system 40, which provides the required level of image magnification and an optical system housing 42. The optical sensor 12, optical sensor case 14, optical system housing 42, and magnifying optical system 40 are positioned in an ordered columnar arrangement along a central axis 314. With respect to the arrangements of Type 1 and Type as shown in FIGS. 3C and 3D, the ordered arrangements are merely reversed. The power management module 36, battery 38, and CPU module 18 may be laterally offset from the central axis 314, filling voids between the ordered columnar arrangement of the optical system and an outer wall of the device 300.

The cartridge 306 includes a stand or posts 316 for supporting the sample and positioning it centrally and at a predetermined distance from the light sources 24a,b and magnifying optical system 40. For Option 1, the cartridge 306a,d includes an object glass 30 where the sample is placed for analysis. The object glass 30 may include an opaque coating 32 on the glass and a sample placement area 34. For Option 2, the cartridge 306c,d may receive a test strip case 44 having a test strip indication area 46. The test strip indication area 46 may include visual elements such as strokes, colors, stripes, or labels to display the results of the analysis.

The first and second housings may be separably connected, for example, by using magnets to hold the housings together and fixing their positions in relation to one another. The first housing 308 may include magnets 26a,b that correspond to magnets 28a,b in the second housing 306. Magnets 26a,b or, alternatively, electrical contacts closing a circuit when magnets 26a,b and coupled to corresponding magnets 28a,b, may also be in communication with the CPU module 18 such that the device can detect, for example, when the housings are separated and rejoined.

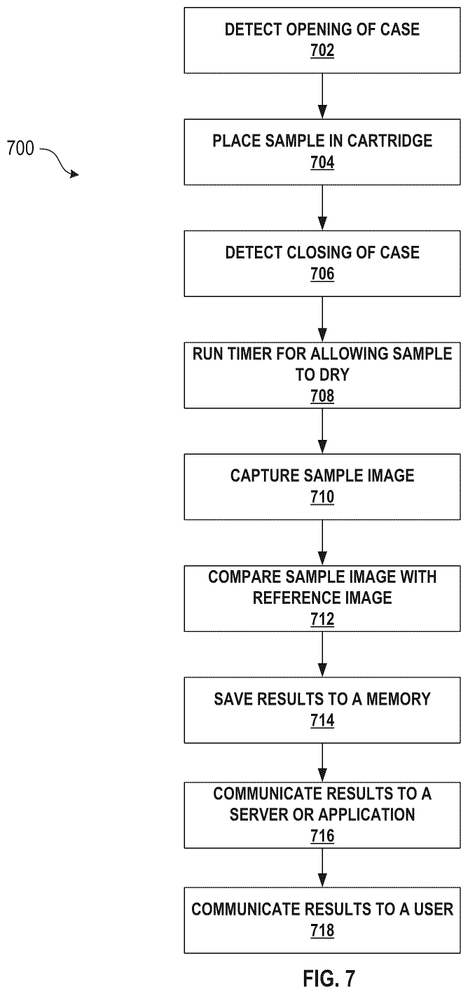

Functionally, the device performs the steps of method 700 as illustrated in FIG. 7: monitor and detect the moment of opening the case 702; monitor and detect the moment the case is closed 706; provide a delay for the time required for the saliva sample to dry 708 (for that test images can be taken); take a contrast image of the saliva sample (for that test images can be taken with different lighting intensity and direction) 710; pre-processes the image and evaluates it 712; save the results to a memory 714; send the results to a cloud service or application via wireless communication interface 716; communicate the results to a user 718. At step 704, a user places a sample in the cartridge. In Option 1 cartridges 306a,c, to speed up the saliva drying process, air holes 310 may provide for air circulation inside of the device.

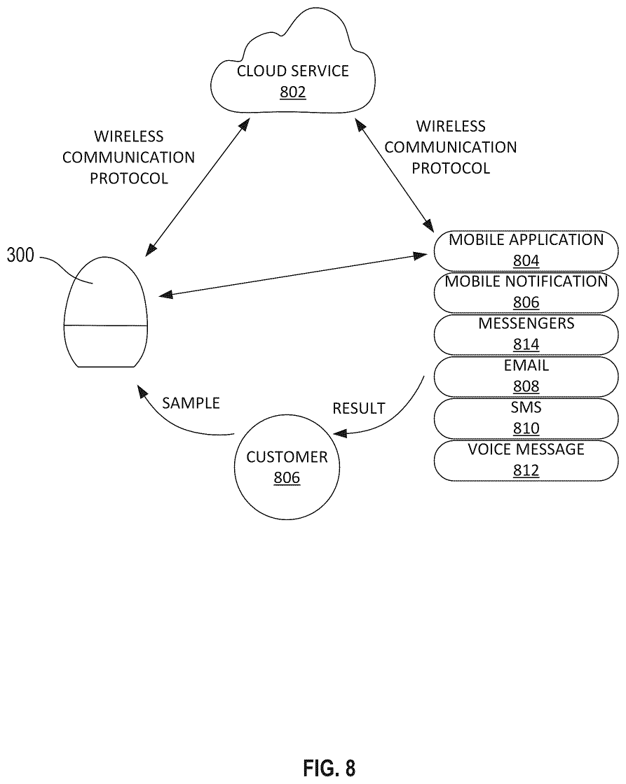

The device 300 has no means of graphically displaying data (results), so communicates with a cloud service 802 and/or mobile application 804 to deliver results to the client, 806 as shown in FIG. 8, which is accessed via a wireless communication protocol, as discussed above, using a unique device identifier. In some embodiments, the device may communicate with the cloud service 802, which, in turn, communications a mobile application 804. In some embodiments, the device 300 may communicate directly with the mobile application 804 which may, in turn, communicate with the cloud service 802. It will be understood that some of the processing or memory functions described herein may take place directly on the device 300, on the cloud service 802, on the mobile application 804, or combinations thereof. For example, results as determined by the device 300 may be stored in the cloud service 802 and could be used, for example, for archiving or tracking purposes. The cloud service 802 may be able to provide updates to the device 300 and/or mobile application 804. Such updates could include, for example, updated or improved reference image data. Result tracking may be used to detect anomalies and alert the user 806. For example, in the case of a sudden change in estrogen levels when no change, or a gradual change, is expected, the device may direct a user 806 to immediately consult with a physician or health care professional for additional diagnostics.

Channels for delivering the result from the cloud service 802 to the client can be selected by the user and are limited only by the available means of communication. Examples of communication channels include, but are not limited to, mobile notifications 806, email messages 808, SMS messages 810, other messenger software and/or protocols 814, and voice messages 812.

The embodiments above are intended to be illustrative and not limiting. Additional embodiments are within the claims. In addition, although the present disclosure has been described with reference to particular embodiments, those skilled in the art will recognize that changes can be made in form and detail without departing from the spirit and scope of the disclosure. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

1. A system configured to determine a physiological state of a woman associated with a hormone level that leads to an appearance of crystals having a characteristic fern shape in a dried sample of a mucous fluid of the woman, comprising:
an optical sensor;
a magnifying optical system;
at least one of a frontal artificial light source and backlit artificial light source;
a diffusion system;
a memory including program code;
processing circuitry configured to execute the program code of the memory, wherein the processing circuitry is configured to:
detect, by the optical sensor, the presence of crystals in a sample by capturing an image of the crystal structure of the sample; and
predict the physiological state of a woman by comparing the crystal density with a reference database; and
an autonomous power management module operatively coupled to the processing circuitry.

2. The system of claim 1, wherein the system further comprises a communications module configured to transmit the predicted physiological state of the woman to a remote device.

3. The system of claim 2, wherein the remote device is a handheld computing device.

4. The system of claim 2, wherein the remote device is a server communicatively coupled to a network.

5. The system of claim 4, wherein the reference database is stored in a memory on the server.

6. The system of claim 2, wherein the remote device is configured to provide an alert indicative of the predicted physiological state to a user.

7. The system of claim 1, wherein the reference database comprises a plurality of images of fern shaped crystals, each of the plurality of images being associated with a predetermined physiological state.

8. The system of claim 1, wherein the physiological state is one of a non-fertile period, a fertile window, or pregnant.

9. A portable and modular analyzer case, in the shape of an egg, for determining a physiological condition comprised of, in combination:
a first housing portion comprising a support for receiving a saliva sample; and
at least a second housing portion which comprises:
an optical sensor;
a magnifying optical system;
a light source;
a diffusion system;
a processing unit; and
a power source,
wherein the light source, diffusion system, magnifying optical system, and optical sensor are disposed in an ordered columnar arrangement along a central vertical axis of the second housing portion such that making the ordered columnar arrangement is disposed by being centered and vertically offset from the saliva sample and able to be managed using hands of the user.

10. The case of claim 9, wherein said first housing portion is separable from the second housing portion, using one hand, along with the entire analyzer case being modular.

11. The case of claim 9, wherein the first and second housing portions may be joined to one another with at least one of a pair of magnets, and the like means for temporarily affixing said housing together.

12. The case of claim 9, wherein the power source is laterally offset from the ordered columnar arrangement.

13. The case of claim 9, wherein the power source comprises at least one of a rechargeable battery, a replaceable battery, and an alternate power source.

14. The case of claim 9, wherein the profile of the case is generally egg shaped, being oblong and able to be balanced within one hand of a user based upon the weight distribution of the unit in the form of an egg.

15. The case of claim 9, wherein the case has a height between 2 to 5 inches, and a width between 1 to 4 inches.

16. The case of claim 9, wherein the support is defined by a test strip, wherein a wall of the first housing portion defines an opening configured to receive therethrough at least a portion of the test strip, and wherein, when the test strip is inserted through the opening, the saliva sample is centered within the second housing portion, within the egg shaped oblong configuration of the unit which is weighted like an egg.

17. A method of determining a physiological state of a woman associated with a hormone level that leads to an appearance of crystals having a characteristic fern shape in a dried sample of a mucous fluid of the woman, wherein the method comprises:
placing a sample of the mucous fluid in an optical path of an optical system of an autonomous device, wherein the optical system comprises an optical sensor, a magnifying optical system, at least one of a frontal artificial light source and a backlit artificial light source, and a diffusion system;
at least partially drying the sample;
obtaining, by the optical system, a contrast image of the at least partially dried sample;
identifying, by processing circuitry operatively coupled to the optical system, based on the contract image, a presence of crystals in the sample;
comparing, by the processing circuitry, the identified crystals to at least one reference image,
predicting, by the processing circuitry, based on the comparison, the physiological state of the woman, and
generating, by the processing circuitry, an alert indicative of the physiological state, wherein the alert is receivable by a human or a machine.

18. The method of claim 17, wherein the method further comprises retrieving, by the processing circuitry, the at least one reference image from a memory operatively coupled to the processing circuitry.

19. The method of claim 17, wherein generating the alert further comprises communicating, by communication circuitry operatively coupled to the processing circuitry, to at least one of a display, a audio device, or a tactile device, the alert, wherein the alert is receivable by the woman.

20. The method of claim 17, wherein the presence of crystals is associated with a level of estrogen.
